(19) United States
(12) Patent Application Publication
(10) Pub. No.: US 2007/0243234 A1
(43) Pub. Date: Oct. 18, 2007

(54) Microscopic Tagging System for Security Identification

(76) Inventors: Peter Gabriele, York, PA (US); Michael Flemmens, Glen Rock, PA (US); Jeffrey Robertson, York, PA (US)

Correspondence Address: BIRCH STEWART KOLASCH & BIRCH, PO BOX 747, FALLS CHURCH, VA, 22040-0747, US

(21) Appl. No.: 11/578,695

(22) PCT Filed: Oct. 31, 2005
(86) PCT No.: PCT/US05/39435

§ 371 Date: Oct. 17, 2006

Related U.S. Application Data
(60) Provisional application No. 60/622,800, filed on Oct. 29, 2004.
(60) Provisional application No. 60/722,011, filed on Sep. 30, 2005.

(51) Int. Cl. A61K 47/00

(52) U.S. Cl. 424/439

(57) ABSTRACT

A microscopic tagging material is provided comprised of a material to which has imparted at least two degrees of identification selected from the group consisting of physical configuration, elemental analysis, functional analysis, and polymeric composition analysis. The material may be a polymeric material, and the tagging material may comprise cut sections of an extruded polymer or polymer composition. The tagging material may be used as an authenticating means for a variety of compositions, coatings and/or products, such as food or drug products.

### BACKGROUND OF THE PRESENT INVENTION

The present invention is directed to a microscopic tagging system for security identification.

The present invention employs micron-sized tags to verify ownership or source of a product or composition by shape identification and/or other associated identification means. Such ownership or source may be determined by tag identity in a film, coating, or composition, or any other material (such as food or pharmaceuticals) where it may be important to verify ownership or any other characteristic of the product (such as exposure to the environment, expiration date, etc.).

Microscopic tagging materials are known as disclosed in U.S. patent publication Nos. 2003/0236219, 2004/0034214, and 2005/0129454. These publications disclose methods of tagging wherein tagging is determined by, for example, the shape or other physical character of the tagging material.

However, it is desirable to provide enhanced levels of security for the tagging material to avoid misuse or counterfeiting of the material.

### DETAIL DESCRIPTION OF THE PRESENT INVENTION

A tagging material is thus provided wherein additional levels of identification security may be incorporated into the tagging material by means of chemical composition (such as the composition of a polymeric material used to form the tagging material), elemental doping of such polymeric material, functional properties, physical configuration, and combinations thereof.

Accordingly, when at least two levels of security are employed, morphology may be the first level of security, while “polymeric fingerprinting” may be the second level of security in the tagging material. An optional third level of security may be “elemental fingerprinting” of the polymeric material. Alternatively, “elemental fingerprinting” may be the second level of security, with the polymer composition being an optional third level of security. The tagging material may be, for example, admixed with any material having rheological properties in the fabrication of a coating or adhesive composition without detriment to the expected physical character of the material to be tagged. Additional levels of security such as functional analysis may also be provided as discussed below.

As discussed above, it is frequently desirable to be able to determine the source and/or identity of compositions, such as hydrocarbon fluids, foodstuffs, pharmaceutical compositions, printing ink, adhesive compositions, etc. Under such circumstances, it is further desirable that the means by which such materials are tagged for identification be unobvious to the naked eye. Verification at low magnification together with shape analysis of a tagging material is one method by which such tagging may occur, as discussed in the above patent publications. However, despite the fact that microscopic shape-sized tagging particles are invisible to the naked eye, shape analysis is not foolproof. Potential counterfeiters can easily copy the shape of such tagging materials and incorporate identical or substantially identical tagging materials into counterfeit compositions.

Additional “levels” of security may thus be desirable or necessary to maintain the desired level of confidence in anti-counterfeiting security as to the determination of identity and/or source of the tagged material.

It may also be desirable to provide tagging means which is functional in character. That is, it may be desirable for the tagging means to also indicate extent of exposure, if any, to deleterious substances such as oxygen, or to establish the “shelf-life” of the tagged material, which may be important with respect to the use of drugs or pharmaceutical compositions.

As noted above, non-shape reliant levels of security as to the tagging material can be provided based on a compositional analysis of the tagging material. Such compositional analysis can occur both by means of the basic composition of, for example, the polymeric material which forms the tagging material, as well as any elemental doping of the polymeric material that is undertaken.

For instance, when a specific polymer blend and/or homo-, co- or terpolymer composition is employed, the identification of the blend or homo-, co- or terpolymer can be confirmed by means of FTIR infrared analysis using the infrared signature or other conventional polymer analytical technique. As to the elemental doping aspect of the present invention, this additional level of identification can be undertaken by means of, for example, electron dispersive analysis or other suitable analytical technique which determines the presence of elemental ions.

Exemplary elemental metals which may be employed to dope the polymer composition which forms the tagging material include but are not limited to elemental iron, tin, lead, platinum, gold, etc., as well as oxides thereof. Advantageously, such metals can all be extruded or drawn, and can also be formed into the shape of a wire for use as a tagging material. Of course, the drawn wire would be small in dimension, and cut into sizes compatible with the size of the tagging material. The elemental material may also be used in the form of fine particles embedded within the tagging material.

The identity of the polymeric material which may be employed as the tagging material is not critical to the present invention. However, it is important for the physical properties of the material to be compatible with the material to be tagged. For instance, if the tagging material is to be added to a composition (such as a polymeric composition) for tagging purposes, the tagging material must be inert in the composition. This is particularly important for drug and pharmaceutical end uses.

If the tagging material is added to the composition prior to any anticipated processing thereof, the tagging material must be able to maintain physical and dimensional stability under the processing conditions. That is, it might be necessary to employ a tagging material which has a higher melting point than any anticipated processing temperature that may be employed.

Of course, a metallic tagging material could also be used alone (without being embedded in a corresponding polymeric material) in such a high temperature environment in the event that multiple levels of security are not required. In such an instance, for example, a polymeric composition could be tagged by the inclusion of particles of a metal or metal alloy in amounts that do not affect the intended physical properties of the polymeric material. Such metallic materials could be used in the form of particles, or alternatively, formed into a specific shape for purposes of detection.

Polymeric materials that may be used to form tagging materials that have physical stability at elevated temperatures include but are not limited to fluoropolymers, polyamides, liquid crystal polymers, polyamideimides, polybenzimidazoles, polyimides, polyketones, polyphenylene sulfides, polysulfones, polyethersulfones, polycyclohexane dimethyl terephthalates, and polycyclohexylene dimethylene terephthalates. As the melting properties of the above polymers vary, the choice of which polymer to use would be determined by the anticipated temperature to be encountered during any processing of the material to be tagged, as well as the intended end use of the material. Such a determination is well within the capability of one of ordinary skill in the art.

To the extent that high temperature properties are not required, a variety of additional polymers may be employed. Such polymers include but are not limited to polyesters, polyethers, polyolefins, thermoplastic polyimides, polycarbonates, polyacrylics, rubbers, polystyrene, polyvinyl acetate, polyvinyl alcohol, polyvinyl chloride, etc. Again, the above listings are merely exemplary and not intended to be all-inclusive by nature.

When employed with foodstuffs or pharmaceutical compositions, the tagging material must be non-toxic and suitable for human consumption As such, any typical food- or pharmaceutical-grade polymeric materials may be so employed. Food- or pharmaceutical-grade polymers are well-known to those of ordinary skill in the art.

U.S. Pat. No. 4,640,035 discloses particulate coding materials comprised of a transverse section of an assembly of elongated elements such as synthetic or natural fibers. Such technology is sometimes referred to as “islands-in-the-sea” technology. The assembly can be produced by, for example, combining pre-existing filaments such as by twisting, or by co-extrusion through a die or spinneret, followed by a draw down step to provide filaments of the desired size, and then transverse sectioning or cutting. The patent teaches that such particulate coding materials may be incorporated into drugs or pharmaceuticals to permit rapid identification in the emergency treatment of overdoses.

However, it has been found advantageous, instead of adding the particulate material to a drug or pharmaceutical composition for identification purposes, to incorporate the drug or pharmaceutical into the particulate coding material itself such that the drug or pharmaceutical would be self-authenticating. For example, the drug or pharmaceutical may be compounded into an edible or bio-compatible polymer which is then co-extruded along with one or more additional polymers to form an assembly of elongated filaments consistent with the teachings of U.S. Pat. No. 4,640,035 directed to the use of islands-in-the-sea technology, herein incorporated by reference. The assembly may then be sectioned or cut into a desired size for use in a pharmaceutical composition together with any desired excipients, fillers, etc. The sectioned or cut pieces may be compounded into a solid tablet, incorporated into a capsule, or administered in liquid form (such as in a syrup, suspension, dispersion, etc.). Indeed, the respective filaments may be extruded through a die or spinneret having holes of differentiatable cross-sectional shape. Even if a drug or pharmaceutical is not compounded into the polymer, other components which provide additional means of authentication consistent with the invention may be added to the polymer prior to extrusion and cutting of the extruded fiber.

U.S. Pat. No. 6,551,353 teaches the preparation of synthetic fibers for medical use by extrusion wherein the fibers contain longitudinal grooves that may contain a medicament to be administered by means of a fabric, etc. formed from the fibers.

It is accordingly possible to confirm the authenticity of the drug or pharmaceutical composition which contains such sectioned or cut pieces by (1) visual inspection (if the authenticating aspect is the cross-sectional shape of either the sectioned or cut assembly, the cross-sectional shape of the drug-containing portion of the assembly, or the color of a portion of the assembly due to the inclusion of a dye or coloring agent), or by physical inspection (depending upon whether any physical characteristics of the polymer(s) used to form the assembly are authenticable, etc). Such authenticating can occur by sampling a portion of the drug or pharmaceutical composition and subjecting the sample to the requisite analysis step.

The tagging materials of the present invention provide a low cost, simple, efficient means for source and/or identity verification. Desirably, the requisite polymer and elemental analysis can be accomplished with conventional laboratory equipment.

The tagging material of the present invention can be employed in many ways. For example, a desired composition of the tagging raw material (such as a specific homo-, co- or terpolymer) can be doped with a specific elemental material. Such doping would generally occur by admixture of the doping material with the polymeric material in melt form. The tags can then be produced from the doped composition in the desired shape by suitable means such as extrusion or melt-spinning of fibers formed of such doped polymers as discussed above. The shape and dimension of the extrusion dye or spinneret determines the physical shape and dimension of the ultimate tagging material. The respective tags may then be cut from the extruded or spun material to the desired dimension or thickness.

The size of such tags may vary with the end use. It is desirable, although not critical, for the tagging material to be of such size such that its presence is not readily apparent in the material to be tagged. As such, the size of the tagging material is desirably within the range of 0.1 to 1.0 mm in its greatest dimension (such as the length of an elongated particle or disk-shaped particle). Such particles would normally have a lesser dimension or thickness in the range of 0.005 to 0.5 mm. To the extent that the shape of the tagging material is to be the first level of security, it is thus desirable for the material to be of such dimension that a particular shape may be practically determined.

For example, a disk-shaped type of tagging material may be used with advantage, with the disk being of any desirable configuration such as circular, rectangular, square-shaped, etc. To aid in the security determination, the disk may have incorporated therein any number of additional security features, such as hole patterns, grooves, etc., that may also be determined upon inspection. For instance, the tagging material may thus be “coded” by a particular combination of a square configuration and pattern of grooves thereon. It is apparent that an infinite number of combinations of “codes” can be imparted to the tagging material, especially if additional levels of security such as polymeric composition and elemental analysis are employed. For instance, the tagging material may include a variety of pre-selected extruded symbols which serve as identifiers, such as numbers or letters, or a differentiable color pattern.

The thus-produced tagging material can be formulated into a composition such as a pressure sensitive adhesive system to “tag” the system as to source and/or identity. Alternatively, the tagging material may be added to materials to be tagged (such as foodstuffs, pharmaceuticals, liquid compositions, etc.) by aerosol, coating and spraying applications, etc. In such an instance, the tagging material could be conveyed in the form of a dispersion together with an inert liquid such as water. By way of further example, it may also be desirable to incorporate tagging materials into the printing ink of ink-jet printers in an attempt to reduce counterfeit product manufacture. The product may also be used in a coating for a drug tablet or on packaging for pharmaceuticals to ensure authenticity of the product.

It is also within the scope of the present invention to provide a tagging material that is chemically or functionally “active”—i.e., the tagging material may undergo either a physical or chemical change when exposed to a pre-determined condition or conditions.

For example, it may be desirable to provide the tagging material with photo-responsive chemistry that will provide a visual effect upon exposure to light such as may be provided by a photocopy machine. Copies made by such a photocopy machine might accordingly be made subject to resolution disruption. This would enable the photocopy to be identified as a photocopy as opposed to an original.

It may further be desirable for the tagging material to have a fixed lifespan, such that after a pre-determined period of time, it can no longer be detected in the tagged material, or the detected characteristic changes based on the passage of time. Such an embodiment could be useful in confirming, for example, the shelf-life of a food or pharmaceutical product.

It may also be important to confirm whether the tagged material has been in contact with any portion of the environment from which it is intended to be isolated. To the extent that a tagged product is to be isolated from oxygen in the air, a tagging material may be employed that includes a component that is reactive with oxygen such that contact with oxygen could be confirmed by a chemical change in the tagging material (color change, chemical change such as by oxidation, etc.). The tagging material could also include a component that is reactive with moisture, such that contact of the tagged material with moisture (if such a result is deemed undesirable) could be confirmed. In such an instance, the security aspect of the invention is not directed so much toward the source or origin of the tagged material, as toward the safety of the material (especially as to foodstuffs and drug or pharmaceutical compositions). The identity of such types of reactive materials would be known to one of ordinary skill in the art.

The tagging material may also exhibit a property that can be determined by conventional analysis, such as radioactivity, luminescence, electrical impedance, fluorescence, etc. Such properties can, of course, be imparted to the tagging material by incorporation of a suitable component if not an inherent property. For instance, a radioactive material (metal or otherwise) can be admixed with a polymeric tagging material to provide a multiple-layered level of security.

1-27. (canceled)

28. A microscopic tagging material suitable for administration of a drug or pharmaceutically-active agent, said microscopic tagging material comprised of a cut transverse section of at least one extruded polymer fiber having a drug or pharmaceutically-active agent embedded therein, said polymer being edible or bio-compatible.

29. (canceled)

30. The tagging material of claim 28, wherein said extruded polymer fiber comprises a composite fiber formed by islands-in-the-sea extrusion.

31. (canceled)

32. The tagging material of claim 30, comprising a sea portion which encompasses at least one island portion which includes said active agent.

33. (canceled)

34. The tagging material of claim 28, wherein said active agent is a pharmaceutically active agent.

35. The tagging material of claim 28, wherein said extruded polymer comprises a pharmaceutical grade excipient.

36. The tagging material of claim 35, wherein said excipient is selected from the group consisting of polylactic acid and cellulose polymers.

37. A drug delivery system comprised of said tagging material of claim 28.

38. The drug delivery system of claim 37 selected from the group consisting of a tablet, capsule, syrup, suspension, ointment, or dispersion.

39. The drug delivery system of claim 39, wherein said tagging material has a cross-sectional configuration which may function as an identifier of source for purposes of identification.

40. The drug delivery system of claim 39, wherein said cross-sectional configuration includes a pre-selected extruded symbol which serves as said identifier.

41. The drug delivery system of claim 40, wherein said pre-selected extruded symbol comprises at least one extruded letter or number.

42. The drug delivery system of claim 40, wherein said pre-selected extruded symbol comprises at least one portion having a differentiatable color portion.

43. (canceled)

44. The tagging material of claim 28, wherein said tagging material has a physical configuration identification means based on a planar configuration of said tagging material.

45. The tagging material of claim 44, wherein said tagging material includes holes or grooves formed in the interior or the periphery of said planar configuration.

46. The tagging material of claim 28, wherein said tagging material includes means for elemental fingerprinting based on the presence of elemental materials in said tagging material.

47. The tagging material of claim 46, wherein said elemental materials comprise one or more metals.

48. The tagging material of claim 28, wherein said tagging material includes means for polymeric fingerprinting based on a composition of a polymeric material that may be present in said tagging material.

49. The tagging material of claim 28, wherein said tagging material includes a degree of identification capable of FTIR infrared analysis.

50. The tagging material of claim 28, wherein said tagging material includes a degree of identification capable of electron dispersive analysis.

51. The tagging material of claim 28, wherein said tagging material includes a degree of identification by fluoresence.

52. The tagging material of claim 28, wherein said tagging material includes a degree of identification by luminescence.

53. The tagging material of claim 28, wherein said tagging material has a pre-determined peripheral configuration together with a pre-determined configuration of holes or voids within the periphery of said tagging material.

54. The tagging material of claim 28, wherein said tagging material has a pre-determined peripheral configuration together with a pre-determined composition within said tagging material comprised of different polymeric materials.

55. The tagging material of claim 28, wherein said tagging material has a pre-determined peripheral configuration together with a pre-determined composition within said tagging material comprised of polymeric materials of differing color.

56. The tagging material of claim 28, wherein said tagging material has a pre-determined peripheral configuration together with a predetermined composition within said tagging material comprised of a polymeric material which is doped with an identifiable non-polymeric material.

57. The tagging material of claim 28, wherein said tagging material includes a component that is functionally active and undergoes a physical or chemical change when exposed to a pre-determined condition or set of conditions.

58. The tagging material of claim 57, wherein said functionally active component is photo-responsive and provides a visual effect upon exposure to a suitable light source.

59. The tagging material of claim 57, wherein said functionally active component has a fixed lifespan.

60. The tagging material of claim 28, wherein the size of said tagging material is within the range of 0.1 to 1 mm in greatest dimension.

61. The tagging material of claim 28, wherein said tagging material has a lesser dimension or thickness within the range of 0.005 to 0.5 mm.

62. The drug delivery system of claim 37, wherein the size of said tagging material is within the range of 0.1 to 1 mm in greatest dimension.

63. The drug delivery system of claim 37, wherein said tagging material has a lesser dimension or thickness within the range of 0.005 to 0.5 mm.
